(19) United States
(12) Patent Application Publication
(10) Pub. No.: US 2022/0146479 A1
(43) Pub. Date: May 12, 2022

(54) MICROFLUIDIC GENERATOR FOR GENERATING A GAS MIXTURE

(71) Applicants: Centre National De La Recherche Scientifique, Paris (FR); In' Air Solutions, Strasbourg (FR); Universite De Strasbourg, Strasbourg (FR)

(72) Inventors: Stéphane LECALVE, Ittenheim (FR); Florian NOEL, Arches (FR); Christophe SERRA, Souffelweyersheim (FR)

(21) Appl. No.: 17/436,402

(22) PCT Filed: Feb. 18, 2020
(86) PCT No.: PCT/EP2020/054244

§ 371 Date: Sep. 3, 2021

(30) Foreign Application Priority Data
Mar. 6, 2019 (FR) FR1902269

(51) Int. Cl. G01N 33/00; G05D 11/13

(52) U.S. Cl. CPC G01N 33/0006; G05D 11/133

(57) ABSTRACT

A method for generating a gaseous mixture by means of an apparatus including at least two inputs, of which the first is an input for a carrier gas and the second is an input for a pollutant and at least one gas output, a system of solenoid valves, a microfluidic circuit and a mixing cell, the microfluidic circuit comprising a sub-circuit that can be isolated or connected with the mixing cell by virtue of the system of solenoid valves is provided. The method includes the following steps: a) cleaning of the microfluidic circuit by pure air received on the first input; b) forming a first air stream with a gas received on the first input of the apparatus, sending of this first air stream to the mixing cell and addition of a pollutant in the sub-circuit isolated from the mixing cell from at least one pollutant received on the second input of the apparatus; and c) opening of the sub-circuit by the system of solenoid valves, so that the sub-circuit is linked to the first input of the apparatus supplied with gas and to the input of the mixing cell, the opening of the sub-circuit provoking the sending of a second air stream to the mixing cell; wherein the steps b) and c) are repeated until the desired quantity of gaseous mixture is obtained at the output of the mixing cell.

### CROSS-REFERENCE TO RELATED APPLICATIONS

This application is a national phase entry of PCT/EP2020/054244, filed on Feb. 18, 2020, which claims the benefit of priority of French Patent Application No. 1902269, filed on Mar. 6, 2019, the contents of which being hereby incorporated by reference in their entirety for all purposes.

### FIELD

The present invention relates to a method for generating a gaseous mixture and a gaseous mixture generator suitable for implementing this method.

### BACKGROUND

Some gaseous mixtures, in particular mixtures containing organic volatile compounds, ammonia (NH₃) or carbon dioxide (CO₂), are used for the calibration of air quality analysis instruments.

These analysis instruments are used to measure the quantity of pollutant in the air. It is therefore necessary to provide them with a gaseous mixture comprising a known concentration of pollutant for the calibration thereof.

Generally, these mixtures are generated from a standard gas diluted in a supplementary gas stream, or by the passage of a pollutant-carrier gas through a temperature-regulated permeation chamber, or from a permeation tube when a diluted mixture is wanted to be produced from an aqueous solution, the carrier gas passing into the permeation tube, or even from a liquid solution injected into an evaporator at 200° C., the vapor of which is then carried off by a carrier gas stream.

The U.S. Pat. No. 5,239,856 describes the generation of standard gas mixtures and, more particularly, a method and an apparatus for generating standard gas mixtures for testing and calibrating highly sensitive analysis instruments such as a mass spectrometer for chemical ionization at atmospheric pressure.

The patent application US 2004/0130965 A1 describes methods for diluting a chemical product used in relation to the fabrication of semiconductor devices and more particularly describes a fixed volume connected between a supply of chemical products and a storage tank.

The drawbacks with these generation methods or apparatuses allowing the generation of these mixtures is that their weight is high and/or that they require high carrier gas flow rates to obtain low concentrations of pollutant in the mixture and/or that they generate high concentrations of pollutant in the mixture. The need to have high flow rates or high concentrations generates significant losses of carrier gas and/or pollutant and, thereby, drastically reduces the autonomy or then requires significant quantities of carrier gas or of pollutant.

Moreover, the weight and therefore the portability of the already existing apparatuses are linked to the weight of the material and/or to the weight of the voluminous bottles of carrier gas (like air) which are essential to generate the necessary carrier gas flow rates (between 1000 and 5000 mL/min). Some apparatuses are nevertheless lightweight but they still require significant carrier gas flow rates.

The invention aims to remedy the abovementioned drawbacks of the prior art, more particularly it aims to propose a method for generating a gaseous mixture and a gaseous mixture generator that can implement the method making it possible to obtain low flow rates, having a low weight and a low carrier gas consumption.

### BRIEF SUMMARY

A subject of the invention is therefore a method for generating a gaseous mixture by means of an apparatus comprising at least two inputs, of which the first is an input for a carrier gas and the second is an input for a pollutant and at least one gas output, a system of solenoid valves, a microfluidic circuit and a mixing cell, the microfluidic circuit comprising a sub-circuit that can be isolated or connected with the mixing cell by virtue of the system of solenoid valves, characterized in that it comprises the following steps:
a) cleaning of the microfluidic circuit by pure air received on the first input;
b) formation of a first air stream with a gas received on the first input of the apparatus, sending of this first air stream to the mixing cell and addition of a pollutant in the sub-circuit isolated from the mixing cell from at least one pollutant received on the second input of the apparatus;
c) opening of the sub-circuit by the system of solenoid valves, so that the sub-circuit is linked to the first input of the apparatus supplied with gas and to the input of the mixing cell, the opening of the sub-circuit provoking the sending of a second air stream to the mixing cell;
the steps b) and c) being repeated until the desired quantity of gaseous mixture is obtained at the output of the mixing cell.

According to embodiments of the invention:
the pollutant added in the isolated sub-circuit during the step b) is gaseous and the step b) also comprises the homogenization of the gaseous pollutant pressure in the sub-circuit; and
the pollutant added during the step b) is liquid and the addition of this pollutant is performed by the deposition of a drop (G) or the sequential deposition of at least two drops of the pollutant in the sub-circuit and, during the step c), the opening of the sub-circuit provokes the evaporation of the drops in the gas coming from the first input of the apparatus.

A second subject of the invention is a gaseous mixture generator for the implementation of the method according to the invention, comprising:
a mass flow rate regulator placed at a first gas input of the generator;
a first 3-way solenoid valve of which the first way is placed at a second gas input of the generator;
a pressure regulator placed between the first way of the 3-way solenoid valve and the second gas input of the generator;
a mixing cell having an input and an output and comprising at least one buffer zone comprising an input and an output, the input of the buffer zone being linked to the input of the cell and the output of the buffer zone being linked to the output of the cell and the output of the cell forming an output of a gaseous mixture from the generator; and
a 6-way solenoid valve of which a first way is linked to an output of the mass flow rate regulator, a second way is linked to the input of the mixing cell, a third way is linked to a second way of the first 3-way solenoid valve, a fourth and fifth ways are linked together and a sixth way is linked to the third way of the 3-way solenoid valve.

According to embodiments of the invention, this gaseous mixture generator can also comprise:
an evaporation cell having a gas input, a liquid input and a gas output; a second and a third 3-way solenoid valves, a first way of the second 3-way solenoid valve being linked to the mass flow rate regulator, a second way of the second 3-way solenoid valve being linked to the first way of the 6-way solenoid valve, a third way of the second 3-way solenoid valve being linked to the gas input of the evaporation cell, a first way of the third solenoid valve being linked to the second way of the 6-way solenoid valve, a second way of the third solenoid valve being linked to the input of the mixing cell and a third way of the third 3-way solenoid valve being linked to the output of the evaporation cell; and a drop generation device placed at the liquid input of the mixing cell and configured so as to form an input of the generator for a liquid;
a drop generation device linked to the fourth way of the 6-way solenoid valve and to the fifth way of the 6-way solenoid valve, and configured so as to form an input of the generator for a liquid;
a drop generation device linked to the third way of the 6-way solenoid valve and to the second way of the first 3-way solenoid valve, and configured so as to form an input of the generator for a liquid; and
a drop generation device chosen from among a syringe, a print head or a microfluidic chip.

A third subject of the invention is a gaseous mixture generator for the implementation of the method according to the invention, comprising:
a mass flow rate regulator placed at a first gas input of the generator;
a first 3-way solenoid valve of which the first way is placed at a second gas input of the generator;
a pressure regulator placed between the first way of the 3-way solenoid valve and the second gas input of the generator;
a mixing cell having an input and an output and comprising at least one buffer zone comprising an input and an output, the input of the buffer zone being linked to the input of the cell and the output of the buffer zone being linked to the output of the cell and the output of the cell forming an output of a gaseous mixture from the generator; and
a 4-way solenoid valve of which a first way is linked to an output of the mass flow rate regulator, a second way is linked to an input of the mixing cell, a third way is linked to a second way of the first 3-way solenoid valve and a fourth way is linked to a third way of the first 3-way solenoid valve.

According to embodiments, this gaseous mixture generator can also comprise:
an evaporation cell having a gas input, a liquid input and a gas output; a second and a third 3-way solenoid valves, a first way of the second 3-way solenoid valve being linked to the mass flow rate regulator, a second way of the second 3-way solenoid valve being linked to the first way of the 4-way solenoid valve, a third way of the second 3-way solenoid valve being linked to the gas input of the evaporation cell, a first way of the third 3-way solenoid valve being linked to the second way of the 4-way solenoid valve, a second way of the third 3-way solenoid valve being linked to the input of the mixing cell and a third way of the third 3-way solenoid valve being linked to the output of the evaporation cell; and a drop generation device placed at the liquid input of the mixing cell and configured so as to form an input of the generator for a liquid;
a drop generation device linked to the third way of the 4-way solenoid valve and to the fourth way of the 4-way solenoid valve and configured so as to form an input of the generator for a liquid; and
a drop generation device chosen from among a syringe, a print head or a microfluidic chip.

A fourth subject of the invention is a gaseous mixture generator for the implementation of the method according to the invention, comprising:
a mass flow rate regulator (RDM) linked to a first input (IN1) of the generator;
a mixing cell (C_MEL) having an input (IN_MEL) and an output (OUT_MEL) and comprising at least one buffer zone (Z1, Z2, Z3, Z4) comprising an input and an output, the input of the buffer zone being linked to the input of the cell and the output of the buffer zone being linked to the output of the cell and the output of the cell forming an output of a gaseous mixture from the generator;
five 2-way solenoid valves (E21, E22, E23, E24, E25);
a pressure regulator (RDP); and
a T coupling (TE),
the output of the mass flow rate regulator being linked to a first way (11, 12) of the first (E21) solenoid valve and of the second (E22) solenoid valve, a second way (21) of the first solenoid valve being linked to a first way (25) of the fifth solenoid valve (E25), a second way (22) of the second solenoid valve (E22) being linked to a first input (T2) of the T coupling, a first way (13) of the third solenoid valve (E23) being linked to a second input of the generator (IN2), a second way (23) of the third solenoid valve (E23) being linked to a second input (T1) of the T coupling, a first way (14) of the fourth solenoid valve and a second way (15) of the fifth solenoid valve (E25) being linked to the input of the mixing cell (C_MEL), a second way (24) of the fourth solenoid valve (E24) being linked to the third input (T3) of the T coupling, the pressure regulator (RDP) being placed between the first way (13) of the third solenoid valve (E23) and the second input of the generator (IN2) and the pressure sensor (P) being placed between the second way (24) of the fourth solenoid valve (E24) and the third input (T3) of the T coupling.

A fifth subject of the invention is a gaseous mixture generator for the implementation of the method according to the invention, comprising:
a mass flow rate regulator (RDM) placed at a first gas input (IN1) of the generator;
a first 3-way solenoid valve (E34) of which the first way (134) is placed at a second gas input (IN2) of the generator;
a pressure regulator (RDP) placed between the first way (134) of the first 3-way solenoid valve and the second gas input of the generator;
a second 3-way solenoid valve (E33) of which the first way (133) is placed at an output of the mass flow rate regulator and the second way (233) is linked to the third way (334) of the first solenoid valve;
a third 3-way solenoid valve (E35) of which the third way (335) is linked to the third way (333) of the second solenoid valve and the first way (135) is linked to the second way (234) of the first solenoid valve; and
a mixing cell (C_MEL) having an input (IN_MEL) and an output (OUT_MEL) and comprising at least one buffer zone (Z1, Z2, Z3, Z4) comprising an input and an output, the input of the buffer zone being linked to the input of the cell, the output of the buffer zone being linked to the output of the cell, the output of the cell forming an output of a gaseous mixture from the generator, and the input of the cell being linked to the second way (235) of the third solenoid valve.

The various gaseous mixture generators according to the invention can comprise:
a pressure sensor (P) configured so as to measure the pressure of pollutant from the second gas input (IN2) of the generator and a computing system (PC) for driving the generator, the computing system being configured to receive as input measurements from the pressure sensor, flow rate values from the mass flow rate regulator (RDM) and pressure values from the pressure regulator (RDP), to control the mass flow rate regulator (RDM) and the pressure regulator (RDP) and to control the openings of the ways of the solenoid valves (E21, E22, E23, E24, E25, E3, E31, E32, E33, E35, E36, E4, E6) of the generator.

In this case:

the computing system can also be configured to control the drop generation device;

the mixing cell can comprise at least two buffer zones (Z1, Z2, Z3, Z4), each of the buffer zones being linked to the input of the mixing cell (IN_MEL) and to the output of the mixing cell (OUT_MEL); and

the mixing cell can be multi-staged, each stage (Et1, Et2, Et3, Et4) of the cell comprising an input (IN_Et1, IN_Et2, IN_Et3, IN_Et4), an output (OUT_Et1, OUT_Et2, OUT_Et3, OUT_Et4) and at least one buffer zone, the input of one stage being linked to the output of another stage, the input of the first stage of the cell being linked to the input of the cell and the output of the last stage of the cell being linked to the output of the cell (OUT_MEL).

### BRIEF DESCRIPTION OF THE SEVERAL VIEWS OF THE DRAWING(S)

Other features, details and advantages of the invention will emerge on reading the description given with reference to the attached drawings which are given by way of example and which represent, respectively:

FIG. 1, a diagram of the steps of the method according to the invention;

FIG. 2a to FIG. 2d, the implementation of the steps of the method of the invention with a first type of gaseous mixture generator for a gaseous pollutant;

FIG. 3a to FIG. 3d, the implementation of the steps of the method of the invention with a second type of gaseous mixture generator for a gaseous pollutant;

FIG. 4, a third type of gaseous mixture generator for the implementation of the method according to the invention for a gaseous pollutant;

FIG. 5a to FIG. 5e, different types of gaseous mixture generator for the implementation of the method according to the invention for a liquid pollutant;

FIG. 6a to FIG. 6c, a fourth type of gaseous mixture generator for the implementation of the method according to the invention for a gaseous pollutant; and

FIG. 7a to FIG. 7e, examples of mixing cells used by the method and/or by the gaseous mixture generators.

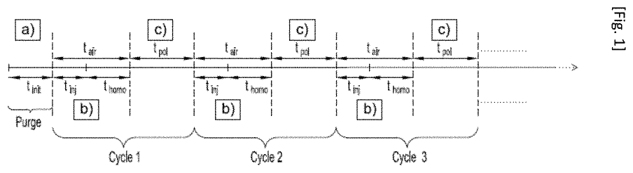

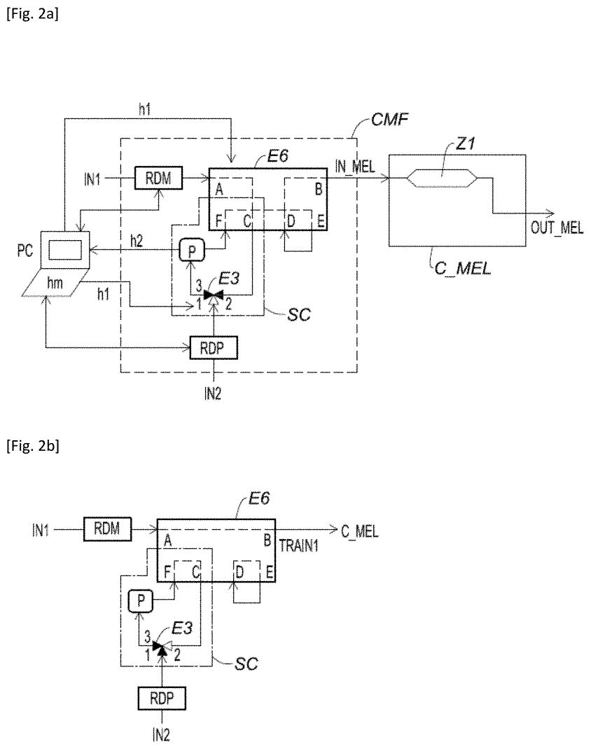

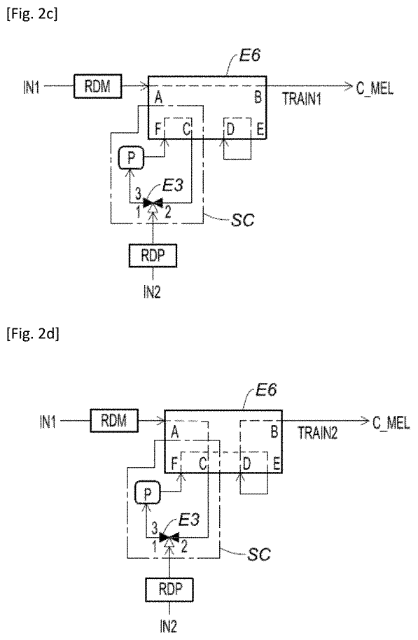

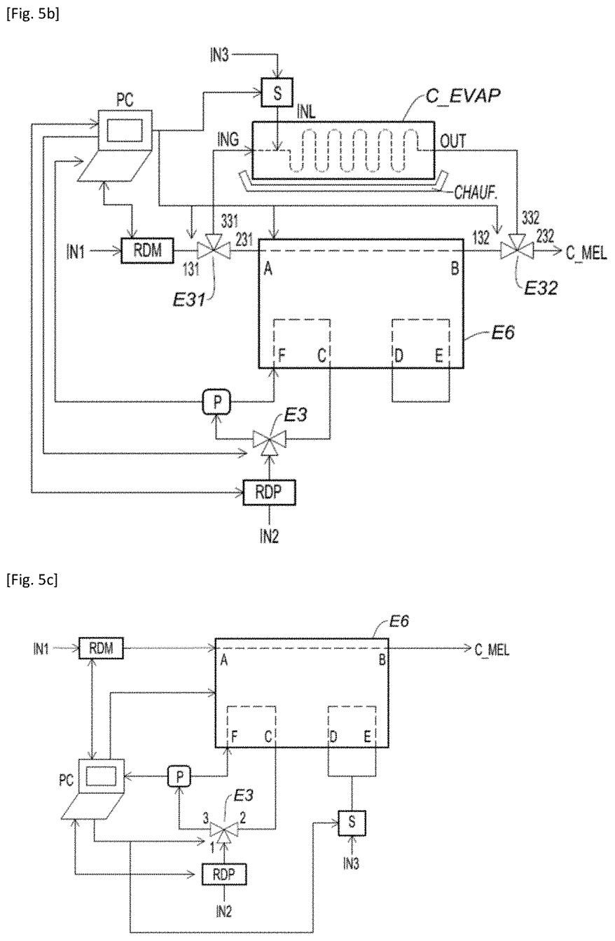

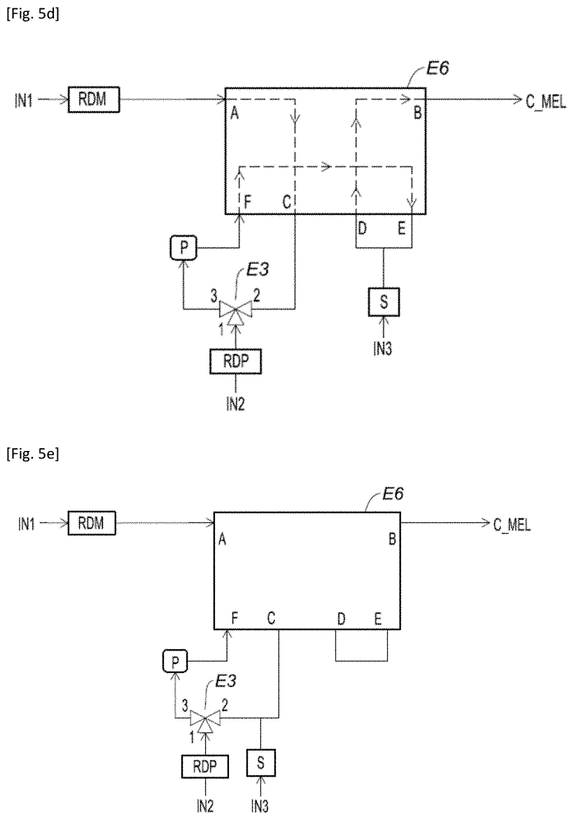

### DETAILED DESCRIPTION

[FIG. 1] presents a diagram of the steps of the method for generating a gaseous mixture according to the invention. The method comprises three steps numbered from a) to c). The steps b) and c) can be repeated several times until the desired quantity of gaseous mixture is obtained. The succession of the steps b) and c) constitutes a cycle, and [FIG. 1] presents three cycles (Cycle 1, Cycle 2, Cycle 3). The method is implemented by means of an apparatus comprising at least two inputs, of which the first is a gas input and the second is an input for a pollutant, at least one gas output, a system of solenoid valves, a microfluidic circuit, itself comprising a sub-circuit and a mixing cell. The sub-circuit can be isolated or connected with the mixing cell by virtue of the system of solenoid valves. Examples of this type of apparatus, called gaseous mixture generator, are described with reference to [FIG. 2a] to [FIG. 5e].

The first step of the method, step a), consists in cleaning the microfluidic circuit of the apparatus with pure air received on the first gas input of the apparatus. This step can also be called Purge. Its duration is denoted tᵢₙᵢₜ.

During the step b), a first air stream is formed with a gas received on the first input of the apparatus and this first air stream is sent to the mixing cell. Preferentially, this first air stream comprises pure air. During the formation and the sending of this first air stream, a pollutant is added in the closed sub-circuit of the apparatus from a pollutant received on the second input of the apparatus. This pollutant can be gaseous, for example an organic volatile compound, or liquid. The duration of this step is denoted tₐᵢᵣ. During the next step, step c), the sub-circuit is opened, so that it is linked to the first gas input of the apparatus and to the input of the mixing cell. That has the effect of stopping the sending of the first air stream to the mixing cell and of provoking the sending of a second air stream including the pollutant to the mixing cell. The duration of the step c) is denoted tₚₒₗ. The total duration of a cycle is therefore equal to t_{cycle}=tₐᵢᵣ+tₚₒₗ.

According to one embodiment, the pollutant added in the step b) is gaseous. In this case, while the first air stream is formed and sent to the mixing cell, the gaseous pollutant is added in the closed sub-circuit for a duration denoted tᵢₙⱼ, then the addition is stopped for the pressure of pollutant to homogenize in the sub-circuit for a duration denoted tₕₒₘₒ. The duration of the step b) is therefore tₐᵢᵣ=tᵢₙⱼ+tₕₒₘₒ.

According to another embodiment, the pollutant added in the step b) is liquid. In this case, the addition of this liquid pollutant is performed by the deposition of a drop or the sequential deposition of at least two drops of the liquid pollutant in the sub-circuit. During the step c), the opening of the sub-circuit provokes the evaporation of the drops in the gas received at the first input of the apparatus. That makes it possible to generate a gaseous mixture from a liquid, for example to generate a gaseous mixture including formaldehyde. The more small drops are generated, the more it will be possible to accurately control the quantity of pollutant injected. The drops generated are smaller than the section of the sub-circuit in which they are injected.

[FIG. 2a] to [FIG. 2d] present a gaseous mixture generator suitable for the implementation of the method according to the invention with a gaseous pollutant, and more particularly the operation of the generator according to the steps of the method.

[FIG. 2a] presents the operation of a generator in the Purge step (step a)). The generator comprises a mass flow rate regulator RDM placed at a first gas input of the generator IN1. This regulator RDM is linked to a first way A of a 6-way solenoid valve E6. The second gas input IN2 of the generator is linked to a first way 1 of a 3-way solenoid valve E3. The third way C of the 6-way solenoid valve E6 is linked to the second way 2 of the 3-way solenoid valve E3 and the third way 3 of the 3-way solenoid valve E3 is linked to the sixth way F of the 6-way solenoid valve E6. A pressure sensor P is placed between the third way 3 of the solenoid valve E3 and the sixth way F of the solenoid valve E6. It makes it possible to measure the pressure of the sub-circuit SC when the latter is closed in the step b) of the method, the sub-circuit SC being formed by the path traveled by the gas, arriving on the second input IN2, in the 3-way solenoid valve E3 and in the sixth F and third C ways of the 6-way solenoid valve. A pressure regulator RDP is placed between the input IN2 and the first way 1 of the solenoid valve E3 to control the quantity of gas injected in the sub-circuit SC. The assembly of 6-way solenoid valve E6, 3-way solenoid valve E3, pressure sensor P, pressure regulator RDP and mass flow rate regulator RDM forms the microfluidic circuit CMF of the generator.

The mass flow rate regulator RDM makes it possible to adapt the flow rate of the gas arriving at the first input IN1 of the generator which makes it possible to send more or less gas into the generator according to the desired quantity and concentration of gas in the gaseous mixture at the output of the generator.

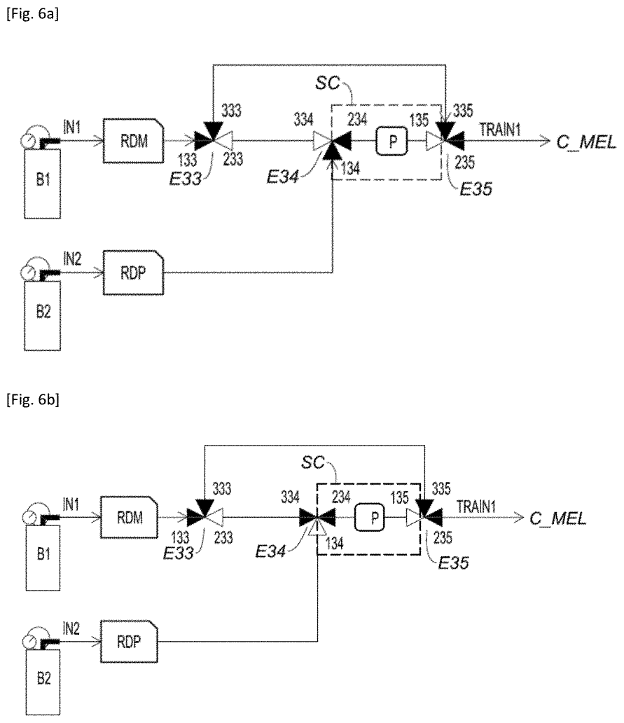

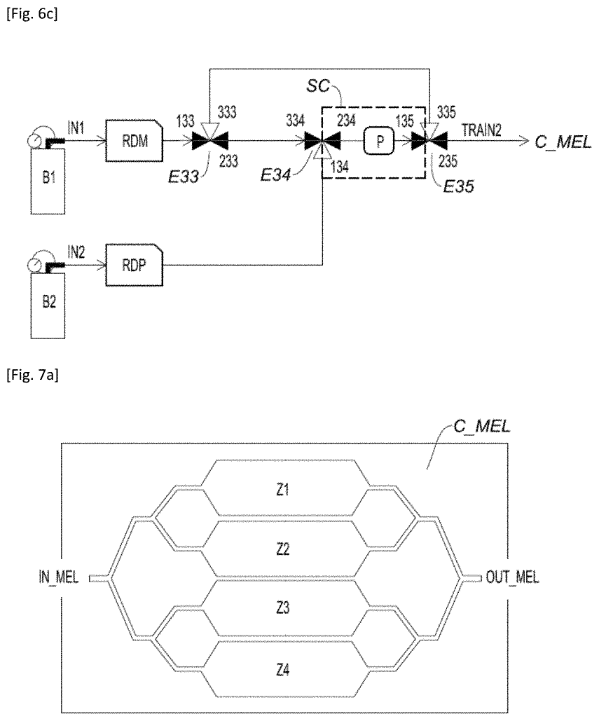

The generator also comprises a mixing cell C_MEL comprising at least one buffer zone Z1 (the cell will be detailed in [FIG. 6a] to [FIG. 6e]). The mixing cell C_MEL comprises an input IN_MEL and a gas output OUT_MEL and its output forms the output of the generator. The second way B of the 6-way solenoid valve E6 is linked to an input of the mixing cell C_MEL.

The fourth D and fifth E ways of the 6-way solenoid valve E6 are linked to one another.

In the step a), inside the 6-way solenoid valve E6, the first way A is linked to the third way C, while the sixth way F is linked to the fifth way E and the fourth way D is linked to the second way B. Furthermore, the first way 1 of the 3-way solenoid valve E3 is closed while its second 2 and third 3 ways are open. That makes it possible to make the gas arriving on the input IN1 of the generator circulate throughout the microfluidic circuit CMF of the generator. Advantageously, the gas sent to the input IN1 is pure air. The gas supplied to this input IN1 can come from a gas bottle or from the previously purified ambient air.

Advantageously, the generator comprises a computing system PC that makes it possible to drive the generator and to receive as input the measurements from the pressure sensor P, the flow rate values from the mass flow rate regulator RDM and the pressure values from the pressure regulator RDP, to control the two regulators RDM and RDP and to control the openings of the ways of the solenoid valves (E6 and E3) of the generator. This computing system PC is detailed after the description of the figures.

[FIG. 2b] and [FIG. 2c] present the operation of the same generator during the step b). During this step, initially ([FIG. 2b]), the first way A of the 6-way solenoid valve E6 is linked to the second way B of the solenoid valve E6. That makes it possible to form and send a first air stream TRAIN1, of the gas coming from the first input IN1 of the generator, to the mixing cell C_MEL. The fourth D and fifth E ways of the 6-way solenoid valve E6 are linked to one another in a closed circuit. The third way C of the solenoid valve E6 is linked to the sixth way F of this same solenoid valve. The second way 2 of the 3-way solenoid valve E3 is closed while the first 1 and third 3 ways of this solenoid valve E3 are open. Thus, the gaseous pollutant arriving on the second input IN2 of the generator is added in the sub-circuit SC, which is closed.

Then, in order to homogenize the pressure of gas coming from the input IN2, the way 1 of the 3-way solenoid valve E3 is closed and the way 2 is opened, the way 3 remaining open. This is represented in [FIG. 2c]. The first air stream TRAIN1 is still sent to the mixing cell C_MEL.

[FIG. 2d] represents the last step of a cycle (step c)) in which the sub-circuit SC is open so that it sends a second air stream TRAIN2 to the mixing cell C_MEL. For that, the inputs 2 and 3 of the 3-way solenoid valve E3 are still open and the first input 1 is closed. The first way A of the 6-way solenoid valve is linked to the third way C while the sixth way F is linked to the fifth way E and the fourth way D is linked to the second way B. Gas is still sent to the first input IN1 of the generator, which has the effect of driving the gas contained in the sub-circuit SC to the mixing cell C_MEL.

The mixing cell C_MEL then receives the air streams TRAIN1 and TRAIN2 one after the other and its function is to mix these two air streams in order, at the output of the mixing cell, for a homogenous gaseous mixture to be obtained that contains both gas of the first air stream TRAIN1 and gas of the second air stream TRAIN2.

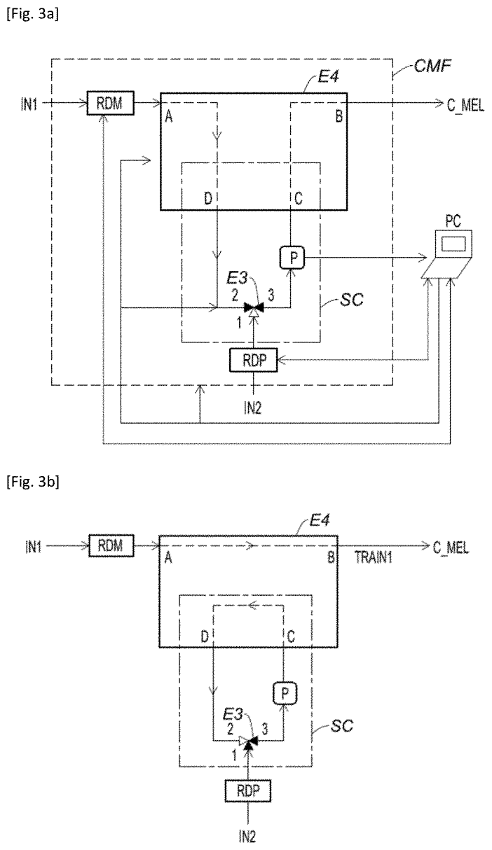

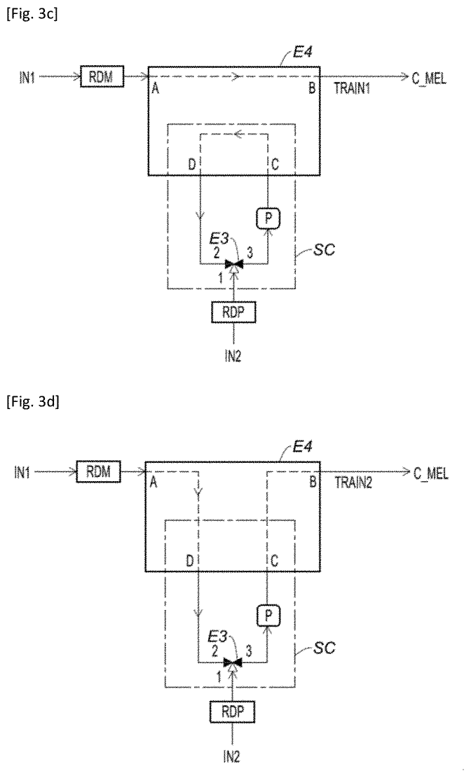

The gas sent to the second input IN2 of the generator is preferentially a volatile organic compound (COV).

[FIG. 3a] to [FIG. 3d] present the implementation of the method according to the invention with a second type of generator.

[FIG. 3a] presents the operation of a second type of generator during the step a), called purge step, of the method. The generator comprises a mass flow rate regulator RDM placed at a first input IN1 of the generator. This regulator RDM is linked to a first way A of a 4-way solenoid valve E4. The second way B of the 4-way solenoid valve E4 is linked to the input of a mixing cell C_MEL included in the generator. The output of the mixing cell C_MEL forms an output of the generator. The first way A of the 4-way solenoid valve is linked to the fourth way D of the solenoid valve E4 and the third way C of E4 is linked to the second way B of the same solenoid valve E4.

A 3-way solenoid valve E3 is also included in the generator. Its first way 1 is linked to a second input IN2 of the generator, while its second way 2 is linked to the fourth way D of the 4-way solenoid valve E4 and its third way 3 is linked to the third way C of the 4-way solenoid valve E4. A pressure sensor P is also present in the generator and is placed between the third way 3 of the solenoid valve E3 and the third way C of the solenoid valve E4. It makes it possible to measure the pressure of gas arriving on the input IN2 of the generator in the sub-circuit SC. The sub-circuit SC is formed by the path traveled by the gas, arriving on the second input IN2, in the 3-way solenoid valve E3 and in the fourth D and third C ways of the 4-way solenoid valve E4. A pressure regulator RDP is placed between the input IN2 and the first way 1 of the solenoid valve E3 to control the quantity of gas injected in the sub-circuit SC.

The assembly of mass flow regulator RDM, pressure regulator RDP, 3- and 4-way solenoid valves (E3, E4) and the pressure sensor P forms the microfluidic circuit CMF of the generator.

During the step a), inside the 4-way solenoid valve E4, the first way A is linked to the fourth way D and the third way C is linked to the second way B. Furthermore, the first way 1 of the 3-way solenoid valve E3 is closed while its second 2 and third 3 ways are open. That makes it possible to make the gas arriving on the input IN1 of the generator circulate throughout the microfluidic circuit CMF of the generator. Advantageously, the gas sent to the input IN1 is pure air. The gas supplied to this input IN1 can come from a gas bottle or from the previously purified ambient air.

As previously, the generator can comprise a computing system PC making it possible to control the generator and to receive as input the measurements from the pressure sensor P and the flow rate or pressure values from the regulators RDM and RDP. This computing system PC is detailed after the description of the figures.

[FIG. 3b] and [FIG. 3c] present the operation of the same generator during the two phases of step b). During this step, initially ([FIG. 3b]), the first way A of the 4-way solenoid valve E4 is linked to the second way B of the solenoid valve E4. That makes it possible to form and send a first air stream TRAIN1, of gas coming from the first input IN1 of the generator, to the mixing cell C_MEL. The third way C of the solenoid valve E4 is linked to the fourth way D of this same solenoid valve. The second way 2 of the 3-way solenoid valve E3 is closed while the first 1 and third 3 ways of this solenoid valve E3 are open. Thus, the gas arriving on the second input IN2 of the generator is added in the sub-circuit SC, which is closed.

Then, in order to homogenize the pressure of gas coming from the input IN2, the way 1 of the 3-way solenoid valve E3 is closed and the way 2 is opened. That is represented in [FIG. 3c]. The first air stream TRAIN1 is still sent to the mixing cell C_MEL.

[FIG. 3d] represents the last step of a cycle (step c)) of the method in which the sub-circuit SC is opened so that it sends a second air stream TRAIN2 to the mixing cell C_MEL. For that, the inputs 2 and 3 of the 3-way solenoid valve E3 are still open and the first input 1 is closed. The first way A of the 4-way solenoid valve E4 is linked to the fourth way D and the third way C is linked to the second way B. Gas is still sent to the first input IN1 of the generator, which has the effect of driving the gas contained in the sub-circuit SC to the mixing cell C_MEL.

The mixing cell C_MEL then receives the air streams TRAIN1 and TRAIN2 one after the other and its function is to mix these two air streams in order, at the output of the mixing cell, to obtain a homogenous gaseous mixture containing both gas of the first air stream TRAIN1 and gas of the second air stream TRAIN2.

As previously, the gas sent to the second input IN2 of the generator is preferentially an organic volatile compound. This gas can be supplied by a bottle or a small pressurized gas tank connected to the second input IN2 of the generator.

According to another embodiment, in the step c), to send gas coming from the first input IN1 of the generator into the sub-circuit SC, the way C of the solenoid valve E4 can be linked to the way A and the way D can be linked to the way B of E4.

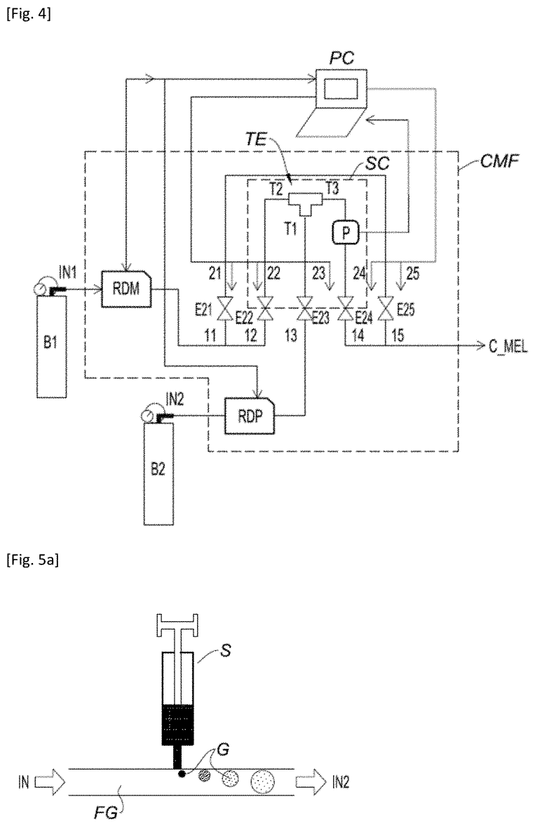

Compared to a generator comprising a 6-way solenoid valve, a generator comprising a 4-way solenoid valve will be less bulky. Nevertheless, the use of a 6-way solenoid valve in the generator makes it possible to consider the generation of a gaseous mixture with two different gases, these gases being the pollutant or a carrier gas including a pollutant.

[FIG. 4] represents a third type of gaseous mixture generator for the implementation of the method according to the invention. This generator comprises a mass flow rate regulator RDM linked to a first input IN1 of the generator. A pressure regulator RDP is linked to a second input IN2 of the generator. The pressure regulator RDP makes it possible to regulate the flow of the gas arriving on the second input IN2 of the generator.

The generator also comprises five 2-way solenoid valves (E21, E22, E23, E24, E25), a T coupling, a pressure sensor and a mixing cell C_MEL. The output of the mass regulator RDM is linked to the first way 11 of the first solenoid valve E21. The second way 21 of the first solenoid valve E21 is linked to the second way 25 of the fifth solenoid valve E25 and the first way 15 of the fifth solenoid valve E25 is linked to the input of the mixing cell C_MEL.

The mass flow regulator RDM is also linked to the first way 12 of the second solenoid valve E22. The first way 14 of the fourth solenoid valve E24 is also linked to the input of the mixing cell C_MEL. The pressure regulator RDP is placed between the second input of the generator IN2 and the first way 13 of the third solenoid valve E23.

The second way 22 of the second solenoid valve E22 is linked to the second input T2 of the T coupling TE, the second way 23 of the third solenoid valve E23 is linked to the first input T1 of the T coupling TE and the second way 24 of the fourth solenoid valve E24 is linked to the third input T3 of the T coupling TE. The pressure sensor P is placed between the second way 24 of the fourth solenoid valve E24 and the third input T3 of the T coupling TE.

During the step a) and the step b), the ways of the following solenoid valves are open, the others being closed:
ways 11 and 21 of the solenoid valve E21;
ways 15 and 25 of the solenoid valve E25;
ways 12 and 22 of the solenoid valve E22;
ways 14 and 24 of the solenoid valve E24; and
ways 13 and 23 of the solenoid valve E23.

Then, to homogenize the pressure of gaseous pollutant, just the two ways 13 and 23 of the solenoid valve E23 are closed.

During the step c), the two ways (12, 22, 14, 24) of the solenoid valves E22 and E24 are open.

Gas bottles (B1, B2) can also be placed at the inputs (IN1, IN2) of the generator in order to supply the generator with gas.

The generator can comprise a computing system PC making it possible to control the generator and to receive as input the measurements from the pressure sensor P and the flow rate and pressure values from the regulators RDM and RDP. This computing system PC is detailed after the description of the figures.

[FIG. 5a] represents a syringe S that can be placed at the input of different ways of the 6-way solenoid valve E6 which makes it possible to deposit one or more drops G of a liquid into an air stream FG. That makes it possible to work with liquid pollutants, such as formaldehyde. For that, a syringe S deposits drops G sequentially into an air stream FG coming from a gas input IN of the generator. The drops G evaporate in the air stream FG, then the air stream FG including the evaporated drops G forms the second air stream sent to the mixing cell. The following figures ([FIG. 5b] to [FIG. 5e]) present different types of generator that make it possible to generate a gaseous mixture from a gaseous pollutant and/or a liquid pollutant.

[FIG. 5b] presents a first type of generator that makes it possible to generate a gaseous mixture from a liquid pollutant. The generator comprises a 6-way solenoid valve E6 and it also comprises two 3-way solenoid valves E31 and E32 and an evaporation cell C_EVAP unlike the generator presented in [FIG. 2a] to [FIG. 2d]. It also comprises a syringe S that makes it possible to introduce a liquid into the generator by the input IN3. The mass flow rate regulator RDM is linked to the first way 131 of the 3-way solenoid valve E31. The second way 231 of the solenoid valve E31 is linked to the first way A of the 6-way solenoid valve E6. The third way 331 of the solenoid valve E31 is linked to a gas input ING of the evaporation cell C_EVAP. The first way 132 of the solenoid valve E32 is linked to the second way B of the solenoid valve E6, its second way 232 is linked to the input of the mixing cell C_MEL and its third way 332 is linked to the output OUT of the evaporation cell C_EVAP. The syringe S is placed so as to be able to deposit drops of a liquid pollutant at the input for a liquid INL of the evaporation cell C_EVAP. The deposited drops then evaporate in the evaporation cell C_EVAP when air, from the first input IN1 of the generator, is sent to the cell C_EVAP. Thus, the mixture comprising the gas from IN1 and the evaporated drops obtained at the output OUT of the evaporation cell C_EVAP forms a second air stream TRAIN2 (such as that of the step c) of the method).

During the formation of the first air stream in the step b), the way 331 of the solenoid valve E31 is open, as is the way 332 of the solenoid valve E32. The ways 231 and 132 of the solenoid valves E31 and E32 are closed. This first air stream TRAIN1 then comprises only the gas entering in the first input IN1 of the generator. Then, during the step c), the ways 331 and 332 remain open and the ways 231 and 132 of the solenoid valves E31 and E32 remain closed, and drops are deposited by virtue of the syringe S in the evaporation cell C_EVAP. The gas then penetrating into the evaporation cell C_EVAP makes it possible to evaporate the drops and thus send a second air stream to the mixing cell C_MEL. This second air stream TRAIN2 comprises the evaporated drops of the liquid pollutant. It is therefore the periodic addition of the drops which makes it possible to alternately generate the two air streams one after the other, without acting on the settings of the solenoid valves E31 and E32.

The evaporation cell C_EVAP can comprise chicanes in order to increase the distance traveled by the mixture, consisting of the drops coming from the input INL and of the gas coming from the input ING, in the evaporation cell. The greater this distance is, the more time the drops have to evaporate in the air stream before reemerging from the cell C_EVAP.

Moreover, in order to accelerate the evaporation of the drops in the evaporation cell C_EVAP, the generator can comprise heating means CHAUF. for heating the evaporation cell C_EVAP. These heating means CHAUF. can for example be chosen from among: a heating ceramic, a heating resistor or an oven, all being able to be insulated to ensure a constant temperature.

The 6-way solenoid valve E6 can be replaced by a 4-way solenoid valve, as in [FIG. 3a] to [FIG. 3d].

The generator can comprise a computing system PC that makes it possible to control the generator, notably the openings of the different ways of the solenoid valves (E3, E31, E32 and E6), the regulators RDP and RDM and the actuator of the syringe S, to receive as input the measurements from the pressure sensor P and the flow rate and pressure values from the regulators RDM and RDP. This computing system PC is detailed after the description of the figures.

[FIG. 5c] and [FIG. 5d] present another type of generator that makes it possible to generate a gaseous mixture from a gaseous and/or liquid pollutant. The generator presented comprises a 6-way solenoid valve as in [FIG. 2a] to [FIG. 2d], and it comprises, in addition, a syringe S linked to the ways D and E of the 6-way solenoid valve E6. A liquid pollutant can thus be deposited in the generator by a third input IN3 of the generator.

[FIG. 5c] presents this generator during the step b) of the method during which the liquid is deposited drop by drop in the closed sub-circuit formed by the ways D and E of the solenoid valve E6. Then, in [FIG. 5d], the sub-circuit is open and the way E is linked to the way F and the way D is linked to the way B of the solenoid valve E6. Thus, the drops deposited during the step b) will evaporate in the air stream arriving from the first input IN1 of the generator and a second air stream TRAIN2 including these evaporated drops is sent to the mixing cell C_MEL.

The generator can comprise a computing system PC making it possible to control the generator, notably the openings of the different ways of the solenoid valves (E3 and E6) and to receive as input the measurements from the pressure sensor P. This computing system PC is detailed after the description of the figures.

The generator described in these two figures can also comprise a heating means, placed in the ways D and E of the solenoid valve E6, making it possible to accelerate the evaporation of the drops in the sub-circuit formed by the ways D and E.

[FIG. 5e] presents yet another type of generator making it possible to generate a gaseous mixture from a liquid or gaseous pollutant. The generator presented comprises a 6-way solenoid valve E6 and a syringe S linked to the way C of this solenoid valve E6. In this example, the solenoid valve E6 could also be a 4-way solenoid valve. As previously, drops of liquid are deposited in the generator by virtue of the syringe S in a sub-circuit. Here, the sub-circuit is formed by the ways F and C of the solenoid valve E6, then, upon the opening of the sub-circuit, the drops evaporate in the air stream coming from the first input IN1 of the generator to form a second air stream TRAIN2. This generator can also comprise a heating means, placed in the ways F and C of the solenoid valve E6, making it possible to accelerate the evaporation of the drops in the sub-circuit formed by the ways F and C.

In these various types of generator, it is perfectly possible to envisage eliminating all the elements that make it possible to receive gaseous pollutant at the input IN2 of the generator and to keep only the elements that make it possible to receive a liquid pollutant by the input IN3 of the generator.

More generally, the syringe S can be replaced by any drop generation device, such as, for example, a print head or a microfluidic chip.

According to another embodiment of the invention, the generators described previously comprise a purification system placed at the first input IN1 of the generator. This purification system makes it possible to purify the air, notably if the gas sent to the input IN1 is ambient air.

[FIG. 6a] to [FIG. 6c] present a fourth type of generator making it possible to generate a gaseous mixture from a gaseous pollutant. The generator comprises three 3-way solenoid valves (E33, E34, E35), a mass flow rate regulator RDM, a pressure regulator RDP, a pressure sensor P and a mixing cell C_MEL. The mass flow rate regulator is placed between a first input IN1 of the generator and the first way 133 of the solenoid valve E33. The first input IN1 is adapted to receive a gas, in particular pure air. A bottle B1 of pure air can thus be placed at the input IN1 of the generator.

The pressure regulator RDP is placed between a second input IN2 of the generator and the first way 134 of the solenoid valve E34. The second input IN2 is adapted to receive a gas, in particular a pollutant gas. A bottle B2 of gaseous pollutant can thus be placed at the input IN2 of the generator.

The pressure sensor P is placed between the second way 234 of the solenoid valve E34 and the first way 135 of the solenoid valve E35.

The second way 233 of the solenoid valve E33 is linked to the third way 334 of the solenoid valve E34. The third way 333 of the solenoid valve E33 is linked to the third way 335 of the solenoid valve E35. The second way 235 of the solenoid valve E35 is linked to the input of the mixing cell C_MEL.

[FIG. 6a] represents the first phase of the step b), that is to say that it takes place during tᵢₙⱼ. The ways 133 and 333 of the solenoid valve E33, the ways 134 and 234 of the solenoid valve E34 and the ways 335 and 235 of the solenoid valve E35 are open, while the other ways are closed. A first pure air stream TRAIN1 is formed and is sent to the mixing cell C_MEL. Pollutant gas is added in the sub-circuit SC, which is closed.

[FIG. 6b] represents the second phase of the step b), that is to say that it takes place during tₕₒₘₒ. The ways 133 and 333 of the solenoid valve E33, the ways 334 and 234 of the solenoid valve E34 and the ways 335 and 235 of the solenoid valve E35 are open, while the other ways are closed. A first pure air stream TRAIN1 is still sent to the mixing cell C_MEL. Gaseous pollutant is no longer added in the sub-circuit SC and the sub-circuit SC is open, which makes it possible for the pressure of pollutant to be homogenized.

[FIG. 6c] presents the step c) of the method, that is to say that it takes place during tₚₒₗ. The ways 133 and 233 of the solenoid valve E33, the ways 334 and 234 of the solenoid valve E34 and the ways 135 and 235 of the solenoid valve E35 are open, while the other ways are closed. Pure air is sent in the sub-circuit SC, which makes it possible to create a second air stream including the gaseous pollutant TRAIN2 which is also sent to the mixing cell C_MEL.

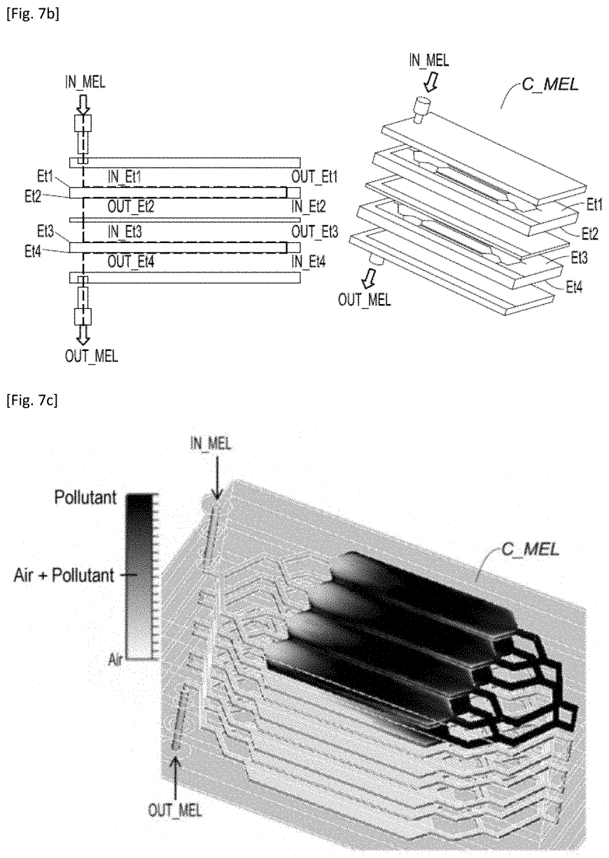

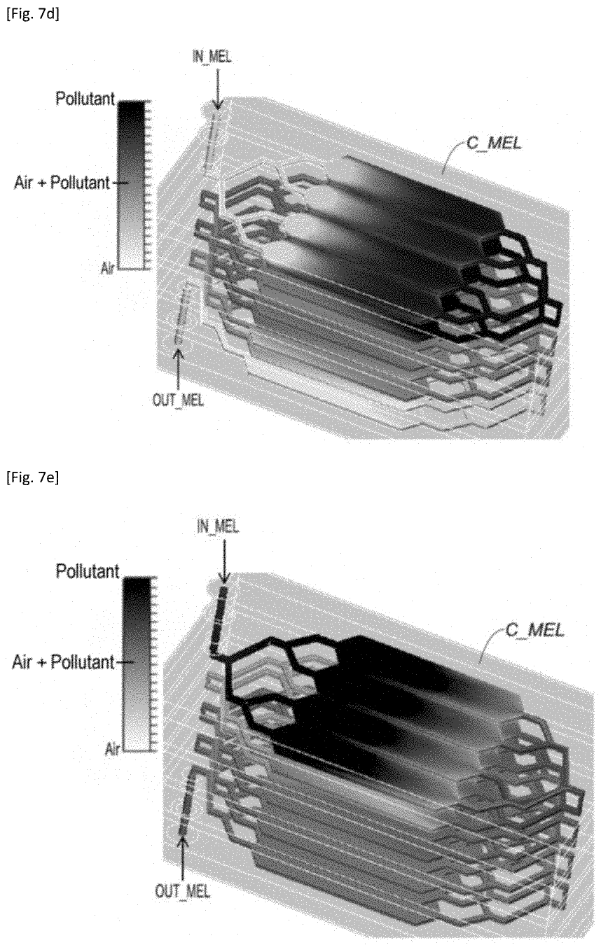

As for the preceding generator configurations, a computing system PC can be present to control the generator, in particular the opening of the ways of the solenoid valves and the two regulators, receive as input the measurements from the pressure sensor P and the flow rate and pressure values from the regulators RDM and RDP.

[FIG. 7a] presents a first example of a mixing cell C_MEL of a generator implementing the method according to the invention. The cell C_MEL comprises an input IN_MEL and an output OUT_MEL. The cell C_MEL comprises at least one buffer zone (Z1, Z2, Z3, Z4) in which the two air streams received are mixed. Advantageously, the cell C_MEL comprises four buffer zones (Z1, Z2, Z3, Z4) placed in parallel, each zone comprising an input linked to the input IN_MEL of the cell C_MEL and an output linked to the output OUT_MEL of the cell C_MEL.

When an air stream enters into the mixing cell C_MEL, it is divided up in the four buffer zones, which makes it possible to promote the homogenous mixing of the two air streams received by the cell C_MEL.

[FIG. 7b] presents a cross-sectional and three-dimensional view of a second example of a mixing cell C_MEL. This cell comprises four stages (Et1, Et2, Et3 and Et4) in which at least one buffer zone is present. Advantageously, on each stage, there are at least two buffer zones placed in parallel in the same way as presented in [FIG. 5a]. The input IN_Et1 of the first stage Et1 is linked to the input IN_MEL of the mixing cell C_MEL and the output OUT_Et4 of the fourth stage Et4 is linked to the output OUT_MEL of the cell C_MEL. The output of an intermediate stage is linked to the input of the next stage.

The multiplying of the buffer zones and/or of the stages in the mixing cell makes it possible to enhance the homogeneity of the gaseous mixture obtained at the output of the mixing cell C_MEL (that is to say, to obtain a gaseous mixture that has a constant concentration of pollutant). The greater the flow rate of gas at the input IN1 of the generator, the more buffer zones and/or stages the cell will comprise. Moreover, if there is a desire not to have multiple stages in the mixing cell, it is also possible to increase the dimensions of the buffer zones (depth and/or width of the zone).

[FIG. 7c] to [FIG. 7e] present the mixing cell C_MEL over time, [FIG. 7c] presenting the cell at t₀, [FIG. 7d] at t₁ and [FIG. 7e] at t₂ with t₀<t₁<t₂. In these three figures, the concentration of pollutant is represented in gray scale: air comprising only the second air stream, here pollutant, can be seen in black in the figures; air comprising only the first air stream TRAIN1, here pure air, can be seen in white in the figures; and air comprising a homogenous mixture of the two air streams can be seen in gray in the figures. At t₀ ([FIG. 7c]), the mixing cell C_MEL receives a second air stream TRAIN2 comprising pollutant, while its output comprises only pure air. At t₁ ([FIG. 7d]), it can be seen that the second air stream is propagated in the cell and that it begins to mix with the pure air already present in the mixing cell (the stages situated in the middle of the cell having become gray). The output of the cell C_MEL still comprises only pure air. A “first air stream” comprising pure air arrives also on the mixing cell at t₁. At t₂ ([FIG. 7e]), the output of the mixing cell C_MEL is a homogenous mixture of the two air streams received.

The buffer zones and input and output channels of the mixing cell can be obtained by etching a substrate, by micro-milling, by three-dimensional printing, or more generally by all the microfluidic circuit production techniques, such as molding or lithography. Furthermore, the materials constituting the mixing cell are inert materials that do not generate any pollutant and that do not react to the contact of the pollutants supplied at the input IN2 or IN3 of the generator. These materials can for example be chosen from among glass, polyetheretherketone (PEEK), polymethyl methacrylate (PMMA) or polytetrafluoroethylene (PTFE).

The generators presented comprise numerous solenoid valves for which there is a need to be able to control the opening and the closing of the ways as accurately as possible in order not to disrupt the cycles and the steps of the method. Thus, the generators can comprise a computing system (represented in figures [FIG. 2a], [FIG. 3a], [FIG. 4], [FIG. 5b] and [FIG. 5c] by the reference PC) which drives the generator, and is configured to receive as input measurements from the pressure sensor, flow rate values from the mass flow rate regulator RDM and pressure values from the pressure regulator RDP, to control the mass flow rate regulator and the pressure regulator and to control the openings of the ways of the solenoid valves of the generator. The computing system PC also controls the drop generation device S.

Thus, the computing system PC is capable of detecting any leaks of gas in the sub-circuit by analyzing the measurements from the sensor, for example before the generator is started up (therefore before the step a)), if the sensor measures pressure variations, or during the step b) or c), if the measured pressure drops suddenly.

This computing system can also make it possible to parameterize and automate the cycles. Furthermore, by virtue of the pressure measurements from the pressure sensor, the system can also calculate the concentration of pollutant of the gaseous mixture at the output of the generator, provided that it is supplied with the concentration of the liquid added drop by drop or the concentration of pollutant supplied at the input of the generator.

Advantageously, the computing system comprises three internal clocks (hm, h1, h2 represented in [FIG. 2a]): a master clock (hm) and two secondary clocks (h1, h2). The purpose of the master clock (hm) is to pace all of the secondary clocks. One of the secondary clocks (h1) makes it possible to drive the solenoid valves, in particular the solenoid valves involved during a gaseous mixture generation cycle, that is to say the valves E3, E4, E6 and E21 to E25, while the other secondary clock (h2) handles the acquisition and the possible display of the measurements from the pressure sensor.

The description of the method and of the generators deals with only one pollutant, but it is perfectly possible to envisage supplying multiple gaseous pollutants with identical or different concentrations depending on the gaseous or liquid sample injected at the input IN2 or IN3 in order to generate a complex gaseous mixture for which it will be possible to calculate the output concentration for each of the pollutants.

1. A method for generating a gaseous mixture by means of an apparatus comprising at least two inputs, of which the first is an input for a carrier gas and the second is an input for a pollutant and at least one gas output, a system of solenoid valves, a microfluidic circuit and a mixing cell, the microfluidic circuit comprising a sub-circuit that can be isolated or connected with the mixing cell by virtue of the system of solenoid valves, the method comprising the following steps:
a) cleaning of the microfluidic circuit by pure air received on the first input;
b) formation of a first air stream with a gas received on the first input of the apparatus, sending of this first air stream to the mixing cell and addition of a pollutant in the sub-circuit isolated from the mixing cell from at least one pollutant received on the second input of the apparatus;
c) opening of the sub-circuit by the system of solenoid valves, so that the sub-circuit is linked to the first input of the apparatus supplied with gas and to the input of the mixing cell, the opening of the sub-circuit provoking the sending of a second air stream to the mixing cell;
wherein the steps b) and c) are repeated until the desired quantity of gaseous mixture is obtained at the output of the mixing cell.

2. The method for generating a gaseous mixture as claimed in claim 1, wherein the pollutant added in the isolated sub-circuit during the step b) is gaseous and the step b) also comprises the homogenization of the gaseous pollutant pressure in the sub-circuit.

3. The method for generating a gaseous mixture as claimed in claim 1, wherein the pollutant added during the step b) is liquid and the addition of this pollutant is performed by the deposition of a drop or the sequential deposition of at least two drops of the pollutant in the sub-circuit and, during the step c), the opening of the sub-circuit provokes the evaporation of the drops in the gas coming from the first input of the apparatus.

4. A gaseous mixture generator for the implementation of the method as claimed in claim 1, comprising:
a mass flow rate regulator placed at a first gas input of the generator;
a first 3-way solenoid valve of which the first way is placed at a second gas input of the generator;
a pressure regulator placed between the first way of the 3-way solenoid valve and the second gas input of the generator;
a mixing cell having an input and an output and comprising at least one buffer zone comprising an input and an output, the input of the buffer zone being linked to the input of the cell and the output of the buffer zone being linked to the output of the cell and the output of the cell forming an output of a gaseous mixture from the generator; and
a 6-way solenoid valve of which a first way is linked to an output of the mass flow rate generator, a second way is linked to the input of the mixing cell, a third way is linked to a second way of the first 3-way solenoid valve, a fourth and a fifth ways are linked together and a sixth way is linked to the third way of the 3-way solenoid valve.

5. The gaseous mixture generator as claimed in claim 4, further comprising:
an evaporation cell having a gas input, a liquid input and a gas output;
a second a third way solenoid valves, a first way of the second 3-way solenoid valve being linked to the mass flow rate regulator, a second way of the second 3-way solenoid valve being linked to the first way of the 6-way solenoid valve, a third way of the second 3-way solenoid valve being linked to the gas input of the evaporation cell, a first way of the third solenoid valve being linked to the second way of the 6-way solenoid valve, a second way of the third solenoid valve being linked to the input of the mixing cell and a third way of the third 3-way solenoid valve being linked to the output of the evaporation cell; and
a drop generation device placed at the liquid input of the mixing cell and configured so as to form an input of the generator for a liquid.

6. The gaseous mixture generator as claimed in claim 4, further comprising a drop generation device linked to the fourth way of the 6-way solenoid valve and to the fifth way of the 6-way solenoid valve, and configured so as to form an input of the generator for a liquid.

7. The gaseous mixture generator as claimed in claim 4, further comprising a drop generation device linked to the third way of the 6-way solenoid valve and to the second way of the first 3-way solenoid valve, and configured so as to form an input of the generator for a liquid.

8. The gaseous mixture generator as claimed in claim 5, wherein the drop generation device is chosen from among a syringe, a print head, or a microfluidic chip.

9. A gaseous mixture generator for the implementation of the method as claimed in claim 1, comprising:
a mass flow rate regulator placed at a first gas input of the generator;
a first 3-way solenoid valve of which the first way is placed at a second gas input of the generator;
a pressure regulator placed between the first way of the 3-way solenoid valve and the second gas input of the generator;
a mixing cell having an input and an output and comprising at least one buffer zone comprising an input and an output, the input of the buffer zone being linked to the input of the cell and the output of the buffer zone being linked to the output of the cell and the output of the cell forming an output of a gaseous mixture from the generator; and
a 4-way solenoid valve of which a first way is linked to an output of the mass flow rate regulator, a second way is linked to an input of the mixing cell, a third way is linked to a second way of the first 3-way solenoid valve and a fourth way is linked to a third way of the first 3-way solenoid valve.

10. The gaseous mixture generator as claimed in claim 9, further comprising:
an evaporation cell having a gas input, a liquid input and a gas output;
a second and a third 3-way solenoid valves, a first way of the second 3-way solenoid valve being linked to the mass flow rate regulator, a second way of the second 3-way solenoid valve being linked to the first way of the 4-way solenoid valve, a third way of the second 3-way solenoid valve being linked to the gas input of the evaporation cell, a first way of the third 3-way solenoid valve being linked to the second way of the 4-way solenoid valve, a second way of the third 3-way solenoid valve being linked to the input of the mixing cell and a third way of the third 3-way solenoid valve being linked to the output of the evaporation cell; and
a drop generation device placed at the liquid input of the mixing cell and configured so as to form an input of the generator for a liquid.

11. The gaseous mixture generator as claimed in claim 9, further comprising a drop generation device linked to the third way of the 4-way solenoid valve and to the fourth way of the 4-way solenoid valve and configured so as to form an input f the generator for a liquid.

12. The gaseous mixture generator as claimed in claim 10, wherein the drop generation device is chosen from among a syringe, a print head or a microfluidic chip.

13. A gaseous mixture generator for the implementation of the method as claimed in claim 1, comprising:
a mass flow rate regulator linked to a first input of the generator;
a mixing cell having an input and an output and comprising at least one buffer zone comprising an input and an output, the input of the buffer zone being linked to the input of the cell and the output of the buffer zone being linked to the output of the cell and the output of the cell forming an output of a gaseous mixture from the generator;
five 2-way solenoid valves;
a pressure sensor;
a pressure regulator; and
a T coupling,
wherein the output of the mass flow rate regulator is linked to a first way of the first solenoid valve and of the second solenoid valve, a second way of the first solenoid valve being linked to a first way of the fifth solenoid valve, a second way of the second solenoid valve being linked to a first input of the T coupling, a first way of the third solenoid valve being linked to a second input of the generator, a second way of the third solenoid valve being linked to a second input of the T coupling, a first way of the fourth solenoid valve and a second way of the fifth solenoid valve being linked to the input of the mixing cell, a second way of the fourth solenoid valve being linked to the third input of the T coupling, the pressure regulator being placed between the first way of the third solenoid valve and the second input of the generator and the pressure sensor being placed between the second way of the fourth solenoid valve and the third input of the T coupling.

14. A gaseous mixture generator for the implementation of the method as claimed in claim 1, comprising:
a mass flow rate regulator placed at a first gas input of the generator;
a first 3-way solenoid valve of which the first way is placed at a second gas input of the generator;
a pressure regulator placed between the first way of the first 3-way solenoid valve and the second gas input of the generator;
a second 3-way solenoid valve of which the first way is placed at an output of the mass flow rate regulator and the second way is linked to the third way of the first solenoid valve;
a third 3-way solenoid valve of which the third way is linked to the third way of the second solenoid valve and the first way is linked to the second way of the first solenoid valve; and
a mixing cell having an input and an output and comprising at least one buffer zone comprising an input and an output, the input of the buffer zone being linked to the input of the cell, the output of the buffer zone being linked to the output of the cell, the output of the cell forming an output of a gaseous mixture from the generator, and the input of the cell being linked to the second way of the third solenoid valve.

15. The gaseous mixture generator as claimed in claim 4, further comprising a pressure sensor configured so as to measure the pressure of pollutant from the second gas input of the generator and a computing system for driving the generator, the computing system being configured to receive as input measurements from the pressure sensor, flow rate values from the mass flow rate regulator and pressure values from the pressure regulator, to control the mass flow rate regulator and the pressure regulator and to control the openings of the ways of the solenoid valves of the generator.

16. The gaseous mixture generator as claimed in claim 15, further comprising a drop generation device placed at a liquid input of the mixing cell and configured so as to form an input of the generator for a liquid, wherein the computing system is also configured to control the drop generation device.

17. The gaseous mixture generator as claimed in claim 4, wherein the mixing cell comprises at least two buffer zones, each of the buffer zones being linked to the input of the mixing cell and to the output of the mixing cell.

18. The gaseous mixture generator as claimed in claim 4, wherein the mixing cell is multi-staged, each stage of the cell comprising an input, an output and at least one buffer zone, the input of one stage being linked to the output of another stage, the input of the first stage of the cell being linked to the input of the cell and the output of the last stage of the cell being linked to the output of the cell.
